(19) United States
(12) Patent Application Publication
(10) Pub. No.: US 2006/0106049 A1
(43) Pub. Date: May 18, 2006

(54) Histone deacetylase inhibitors and methods of use

(75) Inventor: Olatoyosi Odenike, Chicago, IL (US)

Correspondence Address: MICHAEL BEST & FRIEDRICH, LLP, ONE SOUTH PINCKNEY STREET, P O BOX 1806, MADISON, WI, 53701, US

(73) Assignee: The University of chicago, Chicago, IL (US)

(21) Appl. No.: 11/281,666

(22) Filed: Nov. 17, 2005

Related U.S. Application Data
(60) Provisional application No. 60/628,695, filed on Nov. 17, 2004.

(51) Int. Cl. A61K 31/4745

(52) U.S. Cl. 514/291

(57) ABSTRACT

Disclosed are methods of treating an acute myeloid leukemia patient of cytogenetic subgroups having increased histone deacetylase recruitment by administering a histone deacetylase inhibitor to the patient.

### CROSS-REFERENCE TO RELATED APPLICATIONS

This application claims the benefit of U.S. Provisional Application No. 60/628,695, filed Nov. 17, 2004, which is herein incorporated by reference in its entirety.

### STATEMENT REGARDING FEDERALLY SPONSORED RESEARCH OR DEVELOPMENT

This invention was made with government support under CM17102 awarded by the National Institutes of Health. The government has certain rights in the invention.

### INTRODUCTION

Acute myeloid leukemia (AML) constitutes a group of hematopoietic stem cell disorders in which a class of hematopoietic stem or progentitor cells fail to differentiate and over proliferate in the stem cell compartment, leading to an accumulation of non-functional myeloblasts. Hyperproliferation of myeloblasts causes hematopoietic insufficiency (granulocytopenia, thrombocytopenia, and anemia), which results in associated disease symptoms.

Despite recent optimism, improved understanding of the pathophysiology of AML has not resulted in major improvements in the relief of symptoms or survival of people with AML (Stone et al. (2004) Hematology 98-117, which is incorporated by reference in its entirety). Although some patients who undergo aggressive treatment may attain complete remission, a substantial percentage of AML patients are refractory to treatment or experience a relapse following remission.

Aggressive treatment of AML is generally employed to attempt to achieve complete remission because partial remission offers no substantial survival benefit. More than 15% of adults with AML (about 25% of those who attain complete remission) can be expected to survive 3 or more years. Remission rates in adult AML patients are inversely related to age, with an expected remission rate of greater than 65% for those younger than 60 years of age.

There is a need in the art for new methods of treating patients with AML, particularly for those patients who are refractory to treatment or experience a relapse.

### SUMMARY OF THE INVENTION

In one aspect, the present invention provides methods for treating a human with acute myeloid leukemia (AML) characterized by recruitment of histone deacetylase (HDAC) comprising administering to the patient an effective amount of histone deacetylase inhibitor (HDI) or a bioconvertible precursor (or prodrug) to a histone deacetylase inhibitor. The HDI is preferably administered in an amount and for a period of time effective to reduce bone marrow blasts relative to pretreatment bone marrow blast levels or to otherwise achieve a clinical benefit for the patient.

### DETAILED DESCRIPTION

The present invention relates to methods of reducing myeloblasts in a subgroup of people with AML comprising delivering an HDI to a patient in need thereof in an amount effective to ameliorate acute myeloid leukemia.

A substantial number of individuals with AML have been assigned to various cytogenic subgroups based on chromosomal aberrations, including, but not limited to, t(8;21), inv(16), and t(15;17), which correlate with recruitment of HDAC to certain loci. Post-translational modification of histones, which associate with the chromosomes, is believed to affect the degree of DNA coiling. It has been hypothesized that deacetylated histones cause tight coiling, thereby restricting access of transcription factors and RNA polymerase to the DNA, whereas acetylated histones loosen the chromatin structure, thereby permitting gene transcription. The degree of histone acetylation is regulated by histone acetyltransferase (HAT) and HDAC activities. A loss in the balance between the activities of these enzymes may lead to decreased histone acetylation and decreased expression of genes associated with regulation of cell growth.

Deacetylation of histones at those loci causes transcriptional repression and gene silencing, which can result in the proliferation of abnormal cells. In the case of AML, reduced expression of those genes is believed to block differentiation of myelocytes into mature granulocytes (i.e., neutrophils, eosinophils, and basophils).

FK228 (Formula I) is a natural prodrug produced by Chromobacterium violaceum WB968 (FERM BP-1968) that strongly inhibits HDAC in vivo. The isolation and synthesis of FK228 is described in U.S. Pat. No. 4,977,138, which is incorporated by reference in its entirety. Synthetic or semi-synthetic FK228 can be obtained by any suitable means, including the method reported by Khan W. Li, et al. (J. Am. Chem. Soc., Vol. 118, 7237-7238 (1996), which is incorporated by reference in its entirety). The disulfide bond of the prodrug is believed to be reduced to form an active HDI, designated FR135313 (Formula II). In fact, it has been shown that when FK228 is reduced by dithiothreitol in vitro, it forms FR135313, which is capable of inhibiting HDAC (Furumai et al., 2002 Cancer Research 62:4916-4921, which is incorporated by reference in its entirety). 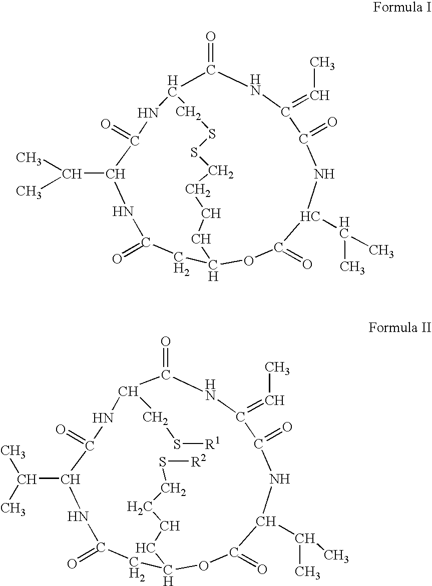
wherein R¹ and R² are the same or different and each is a hydrogen atom or thiol protecting group, or a salt thereof.

FK228 salts include base or acid addition salts such as salts with inorganic base (e.g., alkali metal salts such as sodium salt, potassium salt, and the like, alkaline earth metal salts such as calcium salt, magnesium salt etc., ammonium salt), salts with an organic base (e.g., organic amine salts such as triethylamine salt, diisopropylethylamine salt, pyridine salt, picoline salt, ethanolamine salt, triethanolamine salt, dicyclohexylamine salt, N,N′-dibenzylethylenediamine salt etc.), inorganic acid addition salts (e.g., hydrochloride, hydrobromide, sulfate, phosphate etc.), organic carboxylic acid or sulfonic acid addition salts (e.g., formate, acetate, trifluoroacetate, maleate, tartrate, fumarate, methanesulfonate, benzenesulfonate, toluenesulfonate etc.), salts with a basic or acidic amino acid (e.g., arginine, aspartic acid, glutamic acid etc.) and the like.

Presumably, FK228 is converted to its active reduced form (FR135313) in vivo. It is envisioned that, as an alternative to administering FK228 or its salts to treat AML in patients having the t(8;21) cytogenics, one could practice the method of the invention using FR135313 or its analogs or derivatives, or salts thereof. It is expected that any of a number of suitable analogs of FR135313 having thiol-protecting groups (see U.S. patent application Ser. No. 10/333,063, published as U.S. Publication No. 2004/0053820, which is incorporated by reference in its entirety) would be suitable for use as an HDI or HDI prodrug in the practice of the invention. It is also envisioned that FK228 analogs, such as those described in U.S. Pat. No. 6,403,555 and U.S. Pat. No. 6,548,479, which are incorporated by reference in their entirety, may be suitable for use in the treatment of AML patients having the t(8;21) genotype.

In addition to FK228 and FR135313, a number of other histone deacetylase inhibitors have been described, including, but not limited to, sodium n-butyrate, valproic acid, organic hydroamic acids such as trichostatin A, suberoylanilide hydroxamic acid (SAHA), or LAQ824, trichostatin A, apicidin, trapoxin A, benzamides (e.g., CI=994, MS275), LBH 589, PXD 101, and depsipeptides, the class of compounds to which FK228 belongs. It is envisioned that one or more of these HDIs may be used in the practice of the present invention. Further, salts, esters, other prodrugs, enantiomers, stereoisomers, racemates, polymorphs and the like of these compounds can be administered according to the invention.

A “prodrug” refers to an agent that is converted into a more biologically active form in vivo. Administration of prodrugs may be useful, for example, because of ease of administration. For example, a prodrug may have greater bioavailability by a preferred route of administration than that of the more active form. The prodrug may have greater solubility in pharmaceutical compositions than the more active form of the parent drug.

An example, without limitation, of a prodrug according to the present invention may be administered as an ester (the “prodrug”) to facilitate transmittal across a cell membrane. Another example of a suitable prodrug is a compound of Formula I or II having a short polypeptide, for example, without limitation, a 2-10 amino acid polypeptide, bonded to Formula I or II through its terminal amine group of the polypeptide.

As described in the Examples below, FK228 was administered to patients with refractory or recurring AML by a four hour intravenous infusion at a dose of 13.3 mg/m²/d on days 1, 8, and 15 of a 28-day cycle. At this dosage, patients belonging to cytogenic subgroup t(8;21) exhibited a marked decrease in bone marrow blasts (<5%) and a return to substantially normal hematopoiesis. In addition, a patient having a t(4;21) translocation also responded to the treatment. Both the t(8;21) and t(4;21) cytogenetic subgroups were found to involve the AML1 gene. It is therefore envisioned that the method of the invention may be similarly effective in treating AML in patients having other chromosomal aberrations affecting the AML1 gene.

In the Examples, patients received FK228 at a dose of 13.3 mg/m²/d by intravenous infusion over a four-hour period on days 1, 8, and 15 of a 28-day cycle. Optimal doses may vary according to a number of factors, including the patient's age, size, metabolism, and the like. It is well with the ability of one skilled in the art to optimize dosing. Although dosing at days 1, 8, and 15 in a 28-day cycle afforded good results in certain patient populations, it expected that similar results may be obtained by administering FK228 at different frequencies or intervals over a shorter or longer cycle. Intravenous administration of FK228 is generally in the range of 1 to 1000 mg/day/m² human body surface area, preferably in the range of 5 to 100/md/day/m² human body surface area, and more preferably 10 to 60 mg/day/m² human body surface area by continuous drip infusion administration. In this case, the dose is 0.1 to 100 mg/day/m² human body surface area, preferably 1 to 50 mg/day/m² human body surface area, and more preferably 5 to 30 mg/day/m 2, such as 1 mg/m²/day to about 18 mg/m²/d or about 8.0 to about 15.0 mg/m²/d, human body surface area. The dosing cycle can be repeated one or more times, as necessary. Optimal doses for other HDIs or administration by other routes can be determined employing the above as guidance. An effective amount of an HDI is an amount that achieves a clinical benefit for the patient upon administration and/or an amount which inhibits histone deacetylase in vivo.

It is envisioned that the HDI or HDI prodrug may be administered by any suitable means, including, without limitation, oral, parenteral, intravenous, intramuscular, subcutaneous, implantation, sublingual, buccal, nasal, pulmonary, transdermal, topical, vaginal, rectal, and transmucosal administrations or the like.

Pharmaceutical compositions or preparations according to the present invention contain the HDI (e.g., a compound of Formula I or Formula II), analogs thereof, or a physiologically/pharmaceutically acceptable salt thereof, and may further comprise a physiologically/pharmaceutically acceptable carrier and/or excipient to facilitate administration of the HDI to a patient. The composition or preparation may be a solid, semisolid or liquid preparation (tablet, pellet, troche, capsule, suppository, cream, ointment, aerosol, powder, liquid, emulsion, suspension, syrup, injection etc.) suitable for selected mode of administrating the HDI.

Techniques for formulation and administration of drugs may be found in “Remington's Pharmaceutical Sciences,” (Gennaro A R eds. 2000, which is incorporated by reference in its entirety). Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

The invention further relates to the use of an HDI, such as FK228 or other HDI discussed herein, in the manufacture of a medicament for treating AML in a patient.

It is envisioned that HDI or HDI prodrugs may be used in combination with other therapies, including drug therapies, including, but not limited to, demethylating agents (decitabine, 5azacitidine), clofarabine, fludarabine, cladribine, rituximab (Rituxan), Mylotarg and Gleevec. The HDI can be administered simultaneously with (as a single preparation or as separate preparations), or sequentially to, the other drug therapy. In general, it is envisioned that a combination therapy may include administration of two or more drugs during a single cycle or course of therapy. HDI or HDI prodrugs may be used in combination with non-chemotherapeutic cancer treatments, including radiation and bone marrow transplantation.

In addition to t(8;21), AML patients belonging to other cytogenic subsets correlated with recruitment of histone deacetylase that may be responsive to HDI or HDI prodrugs, either alone or in combination with other drugs, include, but are not limited to, inv 16, t(15;17) and t(4;21), as well as any other chromosomal aberration found to be correlated with histone deacetylase recruitment. Suitably, the patient having AML has a chromosomal aberration affecting the AML1 gene.

Further, patients having refractory AML can be treated according to the invention. A patient having refractory AML is defined herein as a person who has undergone one or more cycles of therapy with an FDA-approved drug (other than FK228) for the treatment of AML and has not experienced a clinically significant response, e.g., has not entered in remission, as that term is commonly understood by persons of ordinary skill in the art of oncology. Patients having a relapse or recurrence of AML can also be treated according to the invention. A patient having an AML relapse or recurrence is defined herein to mean a patient that has undergone one or more cycles of therapy with an FDA-approved drug (other than FK228) for the treatment of AML, has experienced a clinically significant response, e.g., has entered in remission, as that term is commonly understood by persons of ordinary skill in the art of oncology, and has subsequently demonstrated symptoms of AML.

Successful therapy according to the present invention results in the patient receiving a clinical benefit. Such a clinical benefit can include a reduction in bone marrow blasts relative to pretreatment bone marrow blast levels. This reduction can be calculated as a percentage basis of blasts in a relevant tissue sample or peripheral blood. A second clinical benefit can include improved hematopoiesis, such as recovery of substantially normal hematopoiesis, as determined by hematologic analysis and comparison with established normal ranges. In general, these benefits can be determined within 30 days following cessation of HDI therapy or completion of a therapeutic cycle. Other clinical benefits include remission, inhibition of or other decrease in one or more other symptoms of AML, and/or the restoration of one or more normal biological functions in the patient.

The effect of treatment may include one or more of: (1) inhibiting growth of the cancer, i.e., arresting its development, (2) preventing spread of the cancer, i.e., preventing metastases, (3) relieving the cancer, i.e., causing regression of the cancer, (4) preventing recurrence of the cancer, and (5) palliating symptoms of the cancer. “Treatment” refers to therapy, prevention and prophylaxis, and more particularly, refers to the administration of medicine or other modality or to the performance of medical procedures with respect to a patient, for either prophylaxis or to cure or reduce the extent of or likelihood of occurrence of the condition of which the patient is afflicted.

Toxicity and therapeutic efficacy of the compounds described herein can be determined by standard pharmaceutical procedures in cell cultures or experimental animals, e.g., by determining the IC50 and the LD50, wherein the LD50 is the concentration of test compound which achieves a half-maximal inhibition of lethality, for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See e.g., Fingl, et al., “The Pharmacological Basis of Therapeutics”, Ch. 1, p. 1 (1975), which is incorporated by reference in its entirety).

Before any embodiments of the invention are explained in detail, it is understood that all of the compositions and methods disclosed and claimed herein can be made and executed without undue experimentation in light of the present disclosure. While the compositions and methods of this invention have been described in terms of exemplary embodiments, it will be apparent to those skilled in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

All patents and publications listed or described herein are incorporated in their entirety by reference.

Unless defined otherwise, all technical and scientific terms used herein have the same meaning as commonly understood by one of ordinary skill in the art to which this invention belongs.

The following non-limiting Examples are intended to be purely illustrative. The examples are included to demonstrate certain embodiments of the invention. It should be appreciated by those skilled in the art that the methods disclosed in the examples were discovered by the inventors to function well in the practice of the invention, and thus, can be considered to constitute suitable modes for its practice. However, in light of the disclosure, those of skill in the art should appreciate that various changes can be made in the specifically disclosed embodiments without departing from the spirit and scope of the invention.

### EXAMPLES

Eighteen patients (median age=60 years, age range=25-77 years) with relapsed or refractory AML were enrolled in a multicenter Phase II study of depsipeptide for the treatment of AML. Patients were divided into two groups upon entry into the study: Group A (n=14), which included patients without specific chromosomal abnormalities correlated with recruitment of histone deacetylases; and Group B (n=4), which included patients with chromosomal aberrations associated with recruitment of histone deacetylases, such as t(8;21), inv 16, or t(15; 17).

FK228 (Fujisawa, Osaka, Japan) was administered intravenously over four hours at a dose of 13.3 mg/m²/d on days 1, 8, and 15 of a 28-day cycle. Peripheral blood mononuclear cells were obtained prior to (hour 0) and 4 hours (hour 4) and 24 hours (hour 24) after dosing on days 1 and 8 and used to evaluate histone acetylation by flow cytometry and gene re-expression by REAL-time RT-PCR. Target genes of interest include MDR1, a target of HDI-mediated upregulation, and p15^{INK4B} (P15), a target of DNA hypermethylation in AML.

MDR1 and p15 copy numbers were expressed as a normalized quotient of MDR1 and p15, respectively, to the housekeeping gene ABL. The drug was well tolerated, with the most common adverse effects including grade ½ nausea, vomiting, and fatigue.

No objective evidence of response (complete or partial remission) or other evidence of anti-leukemic activity was seen in group A. In contrast, 2 of 4 patients (50%) in Group B exhibited a marked reduction of bone marrow blasts (blast percentage <5%) in the setting of a normocellular marrow after one cycle of treatment, and a concomitant recovery of near-normal hematopoiesis following the second cycle of treatment. This antileukemic effect was short-lived, with both patients exhibiting an increase in bone marrow blasts within 30 days following cessation of therapy. Both patients have translocations involving the AML1 gene. One patient has the t(8;21) cytogenics and the other patient has a novel translocation (4;21). Both of the patients exhibiting chromosomal aberrations and one other patient in the Group B cohort (75%) exhibited an increase in H3 acetylation at 4 and/or 24 hours, whereas only 4 of 14 patients (28%) in Group B exhibited increase H3 acetylation. There was an overall mean increase of 41% in MDR1 expression at hr 4 on days 1 and 8 (p=0.04), and p15 expression showed a mean increase of 91% (p-0.01).

These data suggest that the HDAC inhibitor, FK228, may have anti-leukemic activity in specific cytogenetic subsets of AML known to recruit histone deacetylases, and this is associated with a concomitant increase in histone acetylation. In addition, upregulation of specific target genes occurred in patient derived mononuclear cells, following depsipeptide treatment.

1. A method for treating a patient with acute myeloid leukemia comprising administering to the patient a histone deacetylase inhibitor in an amount effective to reduce bone marrow blasts relative to pretreatment bone marrow blasts, the patient having a chromosomal aberration correlated with increased deacetylation of histone.

2. The method of claim 1, wherein the patient has a chromosomal aberration selected from the group consisting oft(8;21), t(4;21), inv(16), and t(15;17).

3. The method of claim 1, wherein the patient has a chromosomal aberration involving AML1.

4. The method of claim 1, wherein the patient has a chromosomal aberration comprising t(8;21).

5. The method of claim 1, wherein the patient has a chromosomal aberration comprising t(4;21).

6. The method of claim 1, wherein the histone deacetylase inhibitor comprises FK228, FK228 analogs, FR135313, FR135313 analogs, or a pharmaceutically acceptable salt thereof.

7. The method of claim 6, wherein the inhibitor is FK228 or a pharmaceutically acceptable salt thereof.

8. The method of claim 7, wherein the inhibitor is administered in a dose in the range of from about 1 mg/m²/day to about 18 mg/m²/d.

9. The method of claim 7 wherein the inhibitor is administered at a dose in the range of from about 8.0 to about 15.0 mg/m²/d.

10. The method of claim 7, wherein the inhibitor is administered once per week.

11. The method of claim 7, wherein the inhibitor is administered on a 28 day cycle, and wherein the cycle is repeated at least once.

12. The method of claim 1, wherein the patient exhibits substantially normal hematopoiesis.

13. The method of claim 11, wherein the bone marrow blasts are less than 5%.

14. A medicament for treating acute myelogenous leukemia in a patient having a chromosomal aberration correlated with increased histone deacetylation activity comprising FK228, FK228 analogs, FR135313, FR135313 analogs, or salts thereof.
